(19) United States
(12) Patent Application Publication
(10) Pub. No.: US 2023/0390250 A1
(43) Pub. Date: Dec. 7, 2023

(54) MODULATION OF GLUCAGON-LIKE PEPTIDE 1 AND USES THEREOF

(71) Applicant: Robin Elaine DUNCAN, Waterloo (CA)

(72) Inventor: Robin Elaine DUNCAN, Waterloo (CA)

(21) Appl. No.: 18/249,319

(22) PCT Filed: Oct. 29, 2021
(86) PCT No.: PCT/CA2021/051536

§ 371 Date: Apr. 17, 2023

Related U.S. Application Data
(60) Provisional application No. 63/108,184, filed on Oct. 30, 2020.

(51) Int. Cl. A61K 31/42; A61K 45/06; A61K 31/167; A61P 3/04

(52) U.S. Cl. CPC A61K 31/42; A61P 3/04; A61K 31/167; A61K 45/06

(57) ABSTRACT

The present disclosure relates to the modulation of glucagon-like peptide 1 (GLP-1) with inhibitors of lysophosphatidic acid (LPA). LPAR antagonists such as Ki16425, BMS-986020, SAR 100842, AM966, AM095, H2L5186303, LPA2-antagonist 1 and combinations thereof are used in the treatment or prevention of diseases characterized by reduced GLP-1 activity such as: diabetes, Alzheimer's disease, Parkinson's disease, kidney disease (including chronic kidney disease), diabetic nephropathy, a serious renal event, cardiovascular disease, stroke, depression, metal health, pulmonary fibrosis, obesity, aging, or non-alcoholic fatty liver disease.

### FIELD

The present disclosure relates generally to the modulation of glucagon-like peptide 1 (GLP-1), in particular, modulation with inhibitors of lysophosphatidic acid (LPA) receptors and compositions and methods and uses thereof.

### BACKGROUND

Glucagon-like peptide 1 (GLP-1) is a hormone secreted by L-cells of the gastrointestinal tract in response to incoming glucose.

### BRIEF DESCRIPTION OF THE DRAWINGS

Embodiments of the present disclosure will be described, by way of example only, with reference to the Figures herein described.

FIG. 1 shows a proposed mechanism of action whereby LPA inhibits GLP-1 secretion by L-cells.

FIG. 2 shows that various LPA species (2.5 μM) inhibit GLP-1 secretion from cultured GLUTag cells (3.4-fold to 12.5-fold decreases). Secretion was restored by 10 μM Ki-16425 to levels seen in vehicle control wells (all differences between cells treated with LPA only and cells treated with either vehicle alone (0.05% DMSO), or Ki-16425 alone, or Ki-16425 with LPA, were significant (a b Groups with different superscripts are significantly different (P<0.01), while groups sharing the same superscript are not).

FIG. 3 shows that LPA-receptors (LPAR) LPAR1, 2, and 3 are the most abundantly expressed LPAR in GLUTag cells.

FIG. 4 shows that injection of C5761/6J mice with 50 mg/kg of 1-18:1-LPA (i.p.) decreases total plasma GLP-1 levels 30 minutes later by over 60%, and this is partially restored by pre-treating mice 10 min prior to LPA administration with 5 mg/kg (i.p.) of the LPAR 1/2/3 antagonist Ki-16425. n=3. ^{ab}Groups with different superscripts are significantly different P<0.05, while groups sharing the same superscript letter are not.

FIG. 5 shows: plasma LPA concentrations, as determined by mass spectrometry, in male RT-SAKO and control littermate mice, age 16-18 weeks (left panel); and plasma GLP-1 levels in male RT-SAKO mice and control littermates 30 minutes after injection with either Ki-16425 or vehicle control (right panel). *P<0.05 versus measure for the same LPA species in control littermate mice.

FIG. 6 shows that treatment of GLUTag cells for 2.5 h with 2.5 μM 18:2-LPA decreases GLP-1 secretion versus vehicle (0.05% DMSO) or LPAR inhibitor drug alone. LPAR inhibitor drugs do not increase GLP-1 levels alone, but significantly raise GLP-1 secretion from cells also treated with 18:2-LPA, in most cases to levels that are not significantly different from vehicle or drug alone. *P<0.05, ***P<0.001, ****P<0.0001, ns=not significantly different.

FIG. 7 shows an illustrated timeline of the experimental protocol for Example 6.

FIG. 8 shows GLP-1 levels in mice injected with saline (control) or 18:1-LPA and injected with Ki16425 or vehicle control (10% DMSO).

FIG. 9 shows GLP-1 levels in mice injected with saline (control) or 18:1-LPA and gavaged with BMS986020 or vehicle control (10% DMSO).

FIG. 10 shows GLP-1 levels in mice injected with saline (control) or 18:1-LPA and gavaged with SAR100842 or vehicle control (10% DMSO).

FIG. 11 shows GLP-1 levels in mice injected with saline (control) or 18:1-LPA and gavaged with AM966 or vehicle control (10% DMSO).

FIG. 12 shows GLP-1 levels in mice injected with saline (control) or 18:1-LPA and gavaged with AM095 or vehicle control (10% DMSO).

FIG. 13 shows GLP-1 levels in mice injected with saline (control) or 18:1-LPA and gavaged with H2L5186303 or vehicle control (10% DMSO).

FIG. 14 shows GLP-1 levels in mice injected with saline (control) or 18:1-LPA and injected with LPA2-Antagonist 1.

FIG. 15 shows DPP4 gene expression in GLUTag cells with or without treatment with 20:4-LPA after 2 hours of incubation.

FIG. 16 shows DPP4 gene expression in GLUTag cells with or without treatment with 20:4-LPA after 2 days of incubation.

FIG. 17 shows DPP4 activity in GLUTag cells treated with LPA or combinations of LPA and LPAR antagonists.

FIG. 18 shows that DPP4 activity in the serum of C57BI/6J mice is not significantly affected by LPA administration, or administration of Ki16425. Mice were injected with 5 mg/kg Ki16425 or control (10% DMSO in isotonic saline), then injected 10 mins later with 50 mg/kg 1-18:1-LPA or saline (vehicle control), then euthanized 30 minutes later for serum collection and analysis.

FIG. 19 shows that DPP4 activity in the serum of C57BI/6J mice is not significantly affected by LPA administration, or administration of SAR100842. Mice were gavaged with 30 mg/kg SAR100842 or control (10% DMSO in isotonic saline), then injected mins later with 50 mg/kg 1-18:1-LPA or saline (vehicle control), then euthanized 30 minutes later for serum collection and analysis.

FIG. 20 shows that DPP4 activity in the serum of C5761/6J mice is not significantly affected by LPA administration, or administration of AM966. Mice were gavaged with 30 mg/kg AM966 or control (10% DMSO in isotonic saline), then injected 10 mins later with 50 mg/kg 1-18:1-LPA or saline (vehicle control), then euthanized 30 minutes later for serum collection and analysis.

### DETAILED DESCRIPTION

Generally, the present disclosure provides methods of modulating glucagon-like peptide 1 (GLP-1) with modulators of lysophosphatidic acid (LPA) and LPA receptor-mediated signaling.

Glucagon-like peptide 1 (GLP-1) is a hormone secreted by L-cells of the gastrointestinal tract in response to incoming glucose. GLP-1 potentiates insulin secretion by beta-cells of the pancreas, ensuring that adequate amounts of insulin are released to allow for proper disposal of glucose from a meal. GLP-1 levels and action are reduced/impaired in obesity and in Type 2 Diabetes Mellitus (T2DM). People with obesity and T2DM often exist in a state of elevated chronic inflammation. The present disclosure demonstrates that a compound that is produced in inflammation, called lysophosphatidic acid (LPA), can drastically inhibit GLP-1 secretion by L-cells. However, when LPA signaling is blocked using LPA signaling inhibitors, such as LPA-receptor antagonists, the reduction in GLP-1 secretion can be prevented. Therefore LPA-receptor antagonists are expected to be useful for treating conditions where increased GLP-1 is beneficial.

Chronic inflammation in obesity and associated comorbidities (e.g. hepatosteatosis, cardiovascular disease, etc.) elevates circulating LPA, which acts via LPAR1, 2, and 3 to inhibit GLP-1 secretion. Without wishing to be bound by any particular theory, the present disclosure has led to the hypothesis that this occurs through the Gαᵢ-coupled function of LPAR1, 2, and 3 to inhibit adenylyl cyclase (AC), which reduces cAMP levels and intracellular Ca²⁺ concentrations (iCa²⁺), impairing GLP-1 secretion and, thus, glucose-stimulated insulin secretion (GSIS), as shown in FIG. 1.

Type 2 Diabetes Mellitus (T2DM) develops when pancreatic beta-cells no longer secrete enough insulin to fully lower blood glucose after a meal, and it stays chronically high. Beta-cells are stimulated to produce insulin when they encounter a rise in blood glucose. How much insulin they make depends on how high the blood glucose concentration is, and also whether they are exposed to a rise in incretins. Incretins are hormones that the gut secretes to tell the pancreas that glucose is coming, and there are two that are currently known—gastroinhibitory polypeptide (GIP) and glucagon-like peptide 1 (GLP-1). Because they potentiate beta-cell glucose-stimulated insulin secretion, they are called secretagogues. The present disclosure involves the regulation of GLP-1.

Healthy beta-cells can secrete enough insulin to direct the body to dispose of incoming glucose, if those beta-cells are exposed to both glucose and incretins together. However, even healthy beta-cells fail to secrete enough insulin when incretin levels do not rise. They also fail to secrete enough insulin when the beta-cells develop resistance to the action of incretins (i.e. when the GLP-1 receptor (GLP-1R) does not respond properly to GLP-1). There is evidence of an impaired “incretin-effect” in T2DM, with growing evidence of impaired GLP-1R function and overt deficiency in GLP-1 in diabetics and certain overweight populations. In addition to aiding in insulin secretion, GLP-1 also helps to keep beta-cells healthy, protecting them from cell death when they are exposed to high levels of glucose. Thus, having adequate GLP-1 is important for preventing T2DM, and raising GLP-1 levels and/or activity is used to treat T2DM.

Two classes of pharmaceuticals are currently used to improve the efficacy of GLP-1: GLP-1R agonists and dipeptidylpeptidase 4 (DPP4) inhibitors. The first category are drugs, such as Exendin-4, that are synthetic variants of GLP-1, often with modifications that increase the half-life of this compound. DPP4 is the major enzyme responsible for GLP-1 degradation, so inhibition of this enzyme increases endogenous GLP-1 concentrations by preventing breakdown. Together, the success of GLP-1R agonists and DPP4 inhibitors illustrates the clinical significance of raising GLP-1 activity for treatment of T2DM. However, secondary data from clinical trials now indicate benefits of increased GLP-1 on weight loss, cardiovascular disease, diabetic retinopathy and nephropathy, and potentially Alzheimer's disease, among others, that occur independently of effects on glucose lowering or insulin secretion. Raising GLP-1 levels therefore may have benefit in treating a variety of diseases and conditions.

Dozens of dietary compounds, as well as endogenous hormones and metabolites, are known to induce GLP-1 secretion. However, thus far, only two negative regulators of GLP-1 secretion have been identified (i.e. the hormone somatostatin and the neuropeptide galanin). In addition to these, the present disclosure demonstrates that various species of lysophosphatidic acid (LPA) also inhibit GLP-1 secretion from L-cells in culture (FIG. 1, 2, 6), and in mice (FIGS. 4, 5, 8-14). Lysophosphatidic acid (LPA) is a group of bioactive lipids (i.e. fats) that can differ by the type of fatty acyl attached to a glycerophospholipid backbone, and the position (i.e. sn-1 versus sn-2) on that backbone. These species of LPA circulate in the blood and can increase 10-fold when there is infection or inflammation. LPA is produced directly in atherosclerotic plaques, and it contributes to worsening of cardiovascular disease. It also goes up steadily as people become more and more overweight. Inflammation, obesity, and cardiovascular disease are frequently comorbid with Type 2 diabetes mellitus (T2DM). T2DM is considered an inflammatory disease, and there is evidence that inflammation precedes development of T2DM, and also makes it worse. The findings of the present disclosure, namely that LPA inhibits GLP-1 secretion, therefore identifies a new mechanism explaining why obesity and inflammation are risk factors for diseases where GLP-1 is therapeutic, like T2DM, atherosclerosis, kidney disease, and others.

LPA-receptor (LPAR) antagonists tend to be specific to individual LPAR. At present, six bona fide LPAR have been discovered (LPAR1-6) from two different gene families, with LPAR1, 2, and 3 sharing homology, and LPAR 4, 5, and 6 sharing homology. These receptors transduce biological signals in cells from circulating LPA. Different cells have different combinations of LPAR. The LPAR are G-protein coupled receptors (GPCR). Most GPCR have one down-stream effector—for example, they might be coupled to a subunit that will have a stimulatory effect, or an inhibitory effect on cells. LPAR are coupled to at least two, which means that their activation can have different effects in different cells, depending on which pathway is most activated, and this must be determined experimentally. Because the present disclosure has shown that LPA has an inhibitory effect on GLP-1 secretion by L-cells, it is expected that LPA acts in L-cells through an inhibitory G-protein coupling, as illustrated in FIG. 1.

Most LPAR inhibitors target LPAR1, but tend to have some efficacy with LPAR2 or 3, since there is homology between the receptors. The work on LPAR antagonists has focused on conditions in which LPA overproduction is causally implicated, such as the prevention and treatment of fibrosis (e.g. lung, kidney) and on some skin disorders related to inflammation.

A prior study showed that insulin secretion was impaired when mice were injected with LPA, which was prevented by the LPAR1/2/3 antagonist Ki-16425, and that beta-islet cell number is increased in mice given Ki-16425 along with a high-fat diet (Rancoule C, Attane C, Gres S, Fournel A, Dusaulcy R, Bertrand C, et al. Lysophosphatidic acid impairs glucose homeostasis and inhibits insulin secretion in high-fat diet obese mice. Diabetologia. 2013; 56(6):1394-402). However, there was no mention of GLP-1. The teachings of Rancoule et al., and their follow-up review paper (Rancoule et al, Biochimie 96(2014) 140-143), suggests a direct effect of LPA and Ki16425 on beta-islets and insulin secretion by beta-islets, which is counter-intuitive to the results herein disclosed, namely that LPAR regulation might affect other hormones. The present disclosure demonstrates that LPA inhibits GLP-1 secretion, and that this can be abrogated using LPAR inhibitors, therefore identifying the unexpected mechanism responsible. Notably, the present disclosure shows that GLP-1 modulation by LPAR inhibitors have much broader implications, since raising GLP-1 levels can positively impact multiple diseases, beyond (and independent of) effects on glycemic control and insulin regulation.

In the present disclosure, a bioplex assay on kidney ATGL knockout mice (that have higher endogenous LPA) was run, which showed that GLP-1 was significantly decreased, but gastroinhibitory polypeptide (GIP) wasn't. Subsequent blood data herein described suggested that GLP-1 might be modulating the impaired glucose-stimulated insulin secretion. It was particularly surprising that all of this could potentially be explained just by changes in GLP-1, and thus significant further experimentation was warranted.

In the present disclosure, mice were made to have an impairment in kidney fat breakdown (giving them fatty kidneys), which as a byproduct, resulted in the kidneys making more LPA that goes into the blood. What followed was an investigation of GLP-1, but only after the aforementioned bioplex data that showed that GLP-1 was 80% decreased in blood, but GIP was not. The inventors found that Ki16425 rescues the glucose intolerance and impaired insulin secretion that occurs in those mice. It was initially expected (before getting the bioplex data) that this would happen directly through effects on beta-islets; therefore, the inventors isolated them from the mice and cultured them overnight to ‘wash-out’ effects of exposure to blood LPA. Islets from the fatty-kidney mice show a better response to glucose or KCl compared to those from their control littermates. This demonstrated that the islets aren't being significantly impaired by chronic exposure to LPA, once the LPA is removed. Without wishing to be bound by any particular theory, it is possible that the low exposure to GLP-1, and lower-than-needed insulin secretory activity by the islets, and low insulin-environment, is leaving islets primed for GSIS.

Other previous studies teach away from GLP-1 secretion being modulated by LPA modulators. For example, Diakogiannaki et al. (Diabetologica (2013) 56:2688-2696) show that colonocyte cultures from LPAR5 knockout mice have increased GLP-1 secretion in response to the dipeptide glycine-sarcosine, very slightly decreased GLP-1 secretion in response to peptone, and slightly lower GLP-1 secretion in response to forskolin/IBMX that increase cAMP levels to promote secretion (FIG. 6 therein). Their conclusion is that Peptone-stimulated GLP-1 secretion is not dependent on LPAR5.

Thus, the present disclosure demonstrates the unexpected result that LPAR antagonists can also be used to remove a block of GLP-1 secretion caused by elevated LPA. This indicates that LPAR inhibitors are expected to be useful for preventing and treating T2DM, and for improving other disease conditions where increased GLP-1 action has been found to be protective, such as: Alzheimer's disease; Parkinson's disease, chronic kidney disease, kidney disease, diabetic nephropathy, and serious renal events; cardiovascular disease; stroke; depression and mental health; pulmonary fibrosis; obesity; aging, and non-alcoholic fatty liver disease.

In the examples disclosed herein, GLUTag cells were treated with various species of LPA, or Ki16425, or combinations of these; LPA decreased the amount of GLP-1 in the media after 2.5 hours, and Ki16425 co-treatment for 2 hours (beginning 30 minutes after LPA exposure) prevented that decrease. Based on the timeline of only 2 hours, this looks like reduced (and restored) secretion. Without wishing to be bound by any particular theory, while it is possible that reduced (and restored) synthesis could still contribute, in part, to the mechanisms of action of G-protein coupling by LPAR, these results suggest that the change was likely due to secretion. Specifically, it is expected that LPAR coupled to G-alpha-i subunits would inhibit adenylyl cyclase, decreasing cAMP, decreasing intracellular calcium, to decrease the release of GLP-1-containing vesicles when activated by various LPA species, and that antagonism of G-alpha-i coupled LPAR would prevent this.

Based on the present disclosure, there are expected to be benefits to using an LPAR antagonist to increase GLP-1 secretion, as compared to a GLP-1 agonist or a DPP4 inhibitor. GLP-1 agonists and DPP4 inhibitors have side-effects that include nausea, vomiting, diarrhea, headache, weakness and dizziness and nasopharyngitis. Exenatide may worsen kidney disease. Adverse effects of the gliptins (DPP-4 inhibitors) include GI problems (nausea, diarrhea, and stomach pain), flu-like symptoms (headache, runny nose, sore throat) and skin reactions.

GLP-1 affects gastric emptying, so direct administration, or blocking breakdown, can cause the body to ‘over-shoot’ physiological levels. This supra-physiological increase in GLP-1 can have strong GI side effects, and it can take a month or more of medication adjustments to reach a tolerable dose.

As the present disclosure describes, LPAR antagonists act to increase GLP-1 by removing a block that prevents normal regulation. Thus, the mechanism disclosed herein, whereby GLP-1 is increased, is a mechanism that allows the body to act as it normally would. GLP-1 levels are therefore restored by the action of LPAR antagonism, rather than artificially elevated. GLP-1 dosing is therefore physiological, and LPAR antagonism should not result in the side effects associated with excessive GLP-1 levels.

LPA is implicated in multiple pathologies associated with inflammation. Based on the present disclosure, LPAR antagonists could reasonably be expected to have synergistic benefit by both increasing GLP-1 by L-cells, and also by reducing the inflammation that causes the production of LPA that blocks GLP-1 secretion, in the first place.

In one aspect, the present disclosure provides a method of modulating glucagon-like peptide 1 (GLP-1) comprising contacting a cell with a modulator of lysophosphatidic acid (LPA). In one or more embodiments, there is provided a method of modulating glucagon-like peptide 1 (GLP-1) comprising contacting a cell with a modulator of lysophosphatidic acid (LPA) signaling. In some embodiments, the modulator of LPA is a modulator of LPA signaling. In some embodiments, the modulator of LPA is a modulator of LPA activity. In some embodiments, the modulator of LPA is an inhibitor of LPA. In some embodiments the modulator of LPA is a LPA receptor antagonist. Any suitable LPAR antagonist may be usd, such as Ki16425, BMS-986020, SAR 100842, AM966, AM095, H2L5186303, LPA2-antagonist 1 and combinations thereof. In one or more embodiments, the cell is a cell that expresses LPAR and is capable of secreting GLP-1 under suitable conditions. The cell may be an L-cell. The cell may be an alpha cell of the pancreatic islets.

Based on the present disclosure, exemplary diseases and conditions that could be treated with LPAR antagonism through its action of restoring GLP-1 secretion would be those where there is an elevation of LPA (e.g. any inflammatory condition) and where evidence has also been found that GLP-1 may be beneficial in treating that condition, such as those that follow. It will be understood that LPAR antagonism through its action of restoring GLP-1 may be used to treat or prevent any disease or condition characterized by reduced GLP-1 activity.

Diabetes

Based on the present disclosure, it is expected that diabetes could be treated with LPAR antagonism through its action of restoring GLP-1 secretion since decreased GLP-1 secretion and signaling occurs in type 2 diabetes. Elevated LPA is present in type 2 diabetes, which is considered an inflammatory condition. GLP-1 agonism is currently a major way in which diabetes is treated.

Alzheimer's Disease

Based on the present disclosure, it is expected that Alzheimer's disease could be treated with LPAR antagonism through its action of restoring GLP-1 secretion since altered autotaxin-mediated production of LPA in brains of patients with Alzheimer's Disease has been studied, and it has been suggested that blocking LPAR-mediated signaling or autotaxin-mediated LPA production would be therapeutically beneficial. A randomized controlled trial of GLP-1 has found that it prevents declines in brain glucose metabolism, and the positive role of GLP-1 has been discussed.

Chronic Kidney Disease, Kidney Disease, Diabetic Nephropathy, and Serious Renal Events

Based on the present disclosure, it is expected that chronic kidney disease, kidney disease, diabetic nephropathy, and serious renal events could be treated with LPAR antagonism through its action of restoring GLP-1 secretion since LPA is implicated in renal fibrosis and diabetic nephropathy, and LPAR antagonism has been suggested as a therapeutic strategy for treating renal fibrosis. GLP-1 agonists reduce the risk of serious renal events and can act directly in the kidneys, suggesting they have renal benefits beyond glucose lowering.

Cardiovascular Disease

Based on the present disclosure, it is expected that cardiovascular disease could be treated with LPAR antagonism through its action of restoring GLP-1 secretion since LPA is elevated in arterial fibrotic plaques and implicated directly in their formation, while emerging evidence shows that GLP-1 agonism has the potential to stabilize atherosclerotic plaques and reduce arterial inflammation.

Stroke

Based on the present disclosure, it is expected that strokes could be treated with LPAR antagonism through its action of restoring GLP-1 secretion since there may be some evidence that GLP-1 receptor agonism is beneficial for preventing stroke, while LPA is implicated in the pathology of ischemic stroke.

Depression and Mental Health

Based on the present disclosure, it is expected that depression and mental health could be treated with LPAR antagonism through its action of restoring GLP-1 secretion since GLP-1 may have antidepressant effects, while dysregulation of LPA may contribute to, “ . . . many CNS and PNS disorders such as chronic inflammatory or neuropathic pain, glioblastoma multiforme (GBM), hemorrhagic hydrocephalus, schizophrenia, multiple sclerosis, Alzheimer's disease, metabolic syndrome-induced brain damage, traumatic brain injury, hepatic encephalopathy-induced cerebral edema, macular edema, major depressive disorder, stress-induced psychiatric disorder, alcohol-induced brain damage, HIV-induced brain injury, pruritus, and peripheral nerve injury.”

Pulmonary Fibrosis

Based on the present disclosure, it is expected that pulmonary fibrosis could be treated with LPAR antagonism through its action of restoring GLP-1 secretion since inhibiting DPP-4-mediated reduction of GLP-1 has been shown to ameliorate pulmonary fibrosis, while LPA is elevated in, and implicated in, pulmonary fibrosis.

Obesity

Based on the present disclosure, it is expected that obesity could be treated with LPAR antagonism through its action of restoring GLP-1 secretion since GLP-1 receptor agonists are effective for weight loss. LPA is elevated in obese individuals.

Non-Alcoholic Fatty Liver Disease

Based on the present disclosure, it is expected that non-alcoholic fatty liver disease could be treated with LPAR antagonism through its action of restoring GLP-1 secretion since GLP-1 receptor agonists reduce hepatic steatosis, whereas autotaxin activity and LPA are associated with hepatic steatosis.

Parkinson's Disease

Based on the present disclosure, it is expected that Parkinson's disease could be treated with LPAR antagonism through its action of restoring GLP-1 secretion since there is evidence that GLP-1 can have benefits directly on motor control in patients with this disease.

Aging

Based on the present disclosure, it is expected that aging could be treated with LPAR antagonism through its action of restoring GLP-1 secretion. GLP-1 is a candidate for the treatment of impaired glucose tolerance in aging, since older rats maintain their response to GLP-1, and treatment with this incretin can overcome impaired glucose-mediated insulin release. Previous studies have hypothesized that GLP-1 would have a beneficial effect on vascular aging. GLP-1 secretion may be elevated in older adults—but this is hypothesized to counteract a decline in GLP-1 receptor activation (which helps to explain why glucose intolerance worses with age). Because GLP-1 levels may rise, but it's action appears to decrease, this area is evolving. Further study is needed to examine serum levels of GLP-1 in aging, or GLP-1 receptor levels/activation in aging.

LPAR Inhibitors

As disclosed herein, LPAR expression in GLUTag cells has been investigated, and LPAR1, 2, and 3 are most highly expressed, whereas LPAR4, 5, and 6 were expressed at much lower levels or barely detected. Without being bound by any theory, it is expected that LPA is acting through LPAR 1, 2 or 3 (or a combination of these) to inhibit secretion of GLP-1 by L-cells, and that drugs with LPAR1, 2, and/or 3 antagonism activity may relieve that inhibition.

Exemplary LPAR antagonists include but are not limited to:
Ki16425—LPAR1/2/3 antagonist;
BMS-986020—LPAR 1-selective antagonist;
SAR 100842—LPAR1-selective antagonist;
AM966—selective for LPAR1 (with ˜10-fold lower affinity for LPAR2, LPAR3, and lower affinity for LPAR4 and LPAR5);
AM095—selective for LPAR1 (>400-fold greater affinity for LPAR1 than LPAR2-5);
H2L5186303—LPAR2 antagonist (3000 to 5000-fold lower affinity for LPAR1 or LPAR3); and
LPA2-antagonist 1—LPAR 2-selective antagonist.

There are inhibitors of Autotaxin activity (e.g. PF-8380), which could help to reduce circulating LPA. Without wishing to be bound by any theory, it is expected that the effect would be similar to using an LPAR-antagonist, but possibly broader, and with more side effects.

Since there are 6 LPARs, the use of selective antagonists allows one to control which receptors are blocked. If an Autotaxin inhibitor were used, it would be expected to inhibit LPAR 1 to 6. Also, since LPA is often synthesized at sites of injury, LPA synthesis inhibition could impair positive effects of LPA, like wound healing.

GLP-1 Secretion:

There are many implications of increasing or decreasing GLP-1 secretion, apart from activating beta cells to secrete insulin. Disease implications were discussed above.

Side-effects of GLP-1 agonism are discussed above. It is expected that increasing GLP-1 to non-disease-state physiological levels by removing a block, rather than by inducing supraphysiological activity or levels by inhibiting breakdown or consuming agonists, will allow for restored benefits without side effects.

The methods and uses herein may be suitable for modulating GLP-1 in a cell expressing LPAR that is capable of secreting GLP-1 under suitable conditions. The experiments herein disclosed study L-cells in culture. L-cells are thought to be the major source for GLP-1 in circulation. Recently, it is becoming recognized that GLP-1 can also be secreted by alpha cells of the pancreatic islets. This release likely occurs locally (affecting beta cells of the pancreas) and may not be seen in blood measures. Nevertheless, LPA and LPAR antagonists could be of benefit on these cells as well. Accordingly, in one or more embodiments, the cell expressing LPAR that is capable of secreting GLP-1 may be an alpha cell of the pancreatic islets.

### EXEMPLARY EMBODIMENTS

1. A method of modulating glucagon-like peptide 1 (GLP-1) comprising contacting a cell with a modulator of lysophosphatidic acid (LPA).
2. The method of embodiment 1, wherein the modulator of LPA is an inhibitor of LPA signaling or an inhibitor of LPA levels or activity.
3. The method of embodiment 1 or 2, wherein the modulator of LPA is a lysophosphatidic acid receptor (LPAR) antagonist.
4. The method of embodiment 3, wherein the LPAR antagonist is selected from the group consisting of Ki16425, BMS-986020, SAR 100842, AM966, AM095, H2L5186303, LPA2-antagonist 1 and combinations thereof.
The method of embodiment 4, wherein the LPAR antagonist is Ki16425.
6. The method of any preceding embodiment, wherein the cell expresses LPAR and is capable of secreting GLP-1 under suitable conditions.
7. The method of any preceding embodiment, wherein the cell is an L-cell.
8. The method of any preceding embodiment, wherein the contacting takes place in vivo.
9. A method of modulating glucagon-like peptide 1 (GLP-1) in a subject comprising administering to the subject a modulator of lysophosphatidic acid (LPA).
10. The method of embodiment 9, wherein the modulator of LPA is an inhibitor of LPA.
11. The method of embodiment 10, wherein the inhibitor of LPA is a lysophosphatidic acid receptor (LPAR) antagonist.
12. The method of embodiment 11, wherein the LPAR antagonist is selected from the group consisting of Ki16425, BMS-986020, SAR 100842, AM966, AM095, H2L5186303, LPA2-antagonist 1 and combinations thereof.
13. The method of embodiment 12, wherein the LPAR antagonist is Ki16425.
14. The method of any one of embodiments 9 to 12 wherein the subject has or is suspected of having a disease or condition characterized by reduced or impaired GLP-1 levels or activity, glucose homeostasis and/or insulin-stimulated glucose secretion.
A method of increasing glucagon-like peptide 1 (GLP-1) in a subject comprising administering to the subject a therapeutically effective amount of an inhibitor of lysophosphatidic acid (LPA).
16. The method of embodiment 9, wherein the increasing GLP-1 comprises increasing GLP-1 secretion.
17. The method of embodiment 9 or 10, wherein the inhibitor of LPA is a lysophosphatidic acid receptor (LPAR) antagonist.
18. The method of embodiment 11, wherein the LPAR antagonist is selected from the group consisting of Ki16425, BMS-986020, SAR 100842, AM966, AM095, H2L5186303, LPA2-antagonist 1 and combinations thereof.
19. The method of embodiment 12, wherein the LPAR antagonist is Ki16425.
20. The method of any preceding embodiment, wherein the subject is selected to receive the inhibitor of lysophosphatidic acid (LPA) based on a finding or suspicion of reduced or impaired GLP-1 levels or activity.
21. The method of embodiment 20, wherein reduced or impaired GLP-1 levels or activity comprises reduced GLP-1 secretion by L-cells.
22. A method of treating or preventing a disease or condition characterized by reduced GLP-1 levels or activity in a subject, the method comprising administering to the subject a therapeutically effective amount of an inhibitor of lysophosphatidic acid (LPA).
23. The method of embodiment 22, wherein the inhibitor of LPA is an antagonist of lysophosphatidic acid receptor (LPAR).
24. The method of embodiment 23, wherein the LPAR is LPAR-1, LPAR-2, LPAR-3, LPAR-4, LPAR-5, and/or LPAR-6.
25. The method of embodiment 24, wherein the LPAR is LPAR-1, LPAR-2, and/or LPAR-3.
26. The method of embodiment 23, wherein the LPAR antagonist is selected from the group consisting of Ki16425, BMS-986020, SAR 100842, AM966, AM095, H2L5186303, LPA2-antagonist 1 and combinations thereof.
27. The method of embodiment 26, wherein the lysophosphatidic acid receptor (LPAR) antagonist is Ki16425.
28. The method of any one of embodiments 22 to 27, wherein the disease or condition is diabetes, Alzheimer's disease, Parkinson's disease kidney disease (including chronic kidney disease), diabetic nephropathy, a serious renal event, cardiovascular disease, stroke, depression, metal health, pulmonary fibrosis, obesity, aging, or non-alcoholic fatty liver disease.
29. The method of embodiment 28, wherein the disease or condition is obesity.
30. The method of embodiment 28, wherein the disease or condition is Type II diabetes.
31. The method of embodiment 28, wherein the disease or condition is aging.
32. The method of any one of embodiments 22 to 27, wherein reduced GLP-1 levels or activity comprises decreased GLP-1 secretion, decreased GLP-1 production, decreased GLP-1 sensitivity, decreased GLP-1 receptor levels or binding, increased GLP-1 breakdown, excessive GLP-1 inhibition, or a combination thereof.
33. The method of embodiment 32, wherein reduced GLP-1 activity comprises decreased GLP-1 secretion from L-cells.
34. The method of any one of embodiments 22 to 33, which further comprises administration of a DPP4 inhibitor.
35. The method of any one of embodiments 22 to 33, which further comprises administration of a GLP-1 agonist.
36. The method of any one of embodiments 22 to 34, which further comprises administration of another GLP-1 regulator.
37. A method of selecting a subject for treatment with an inhibitor of lysophosphatidic acid (LPA) comprising assessing GLP-1 levels or activity in the subject and, if the GLP-1 levels or activity is lower than desired, selecting the subject for the treatment.
38. The method of embodiment 37, wherein the inhibitor of LPA is an antagonist of lysophosphatidic acid receptor (LPAR).
39. The method of embodiment 38, wherein the LPAR is LPAR-1, LPAR-2, LPAR-3, LPAR-4, LPAR-5, and/or LPAR-6.
The method of embodiment 39, wherein the LPAR is LPAR-1, LPAR-2, and/or LPAR-3.
41. The method of embodiment 38, wherein the LPAR antagonist is selected from the group consisting of Ki16425, BMS-986020, SAR 100842, AM966, AM095, H2L5186303, LPA2-antagonist 1 and combinations thereof.
42. The method of embodiment 41, wherein the lysophosphatidic acid receptor (LPAR) antagonist is Ki16425.
43. The method of any one of embodiments 37 to 42, wherein the subject has or is suspected of having a disease or condition characterized by reduced or impaired GLP-1 levels or activity, glucose homeostasis and/or insulin-stimulated glucose secretion.
44. The method of any one of embodiments 37 to 43, wherein the subject has or is suspected of having a disease or condition selected from the group consisting of diabetes, Alzheimer's disease, Parkinson's disease, kidney disease (including chronic kidney disease), diabetic nephropathy, a serious renal event, cardiovascular disease, stroke, depression, metal health, pulmonary fibrosis, obesity, aging, or non-alcoholic fatty liver disease.
45. The method of embodiment 44, wherein the disease or condition is obesity.
46. The method of embodiment 44, wherein the disease or condition is Type II diabetes.
47. The method of embodiment 42, wherein the disease or condition is aging.
48. The method of any one of embodiments 22 to 27, wherein reduced GLP-1 levels or activity comprises decreased GLP-1 secretion, decreased GLP-1 production, decreased GLP-1 sensitivity, decreased GLP-1 receptor levels or binding, increased GLP-1 breakdown, excessive GLP-1 inhibition, or a combination thereof.
49. Use of an LPAR antagonist for treating or preventing a disease or condition that responds to GLP-1 therapy.
50. Use of an LPAR antagonist for treating or preventing a disease or condition characterized by reduced GLP-1 activity.
51. Use of an LPAR antagonist for increasing GLP-1 levels or activity in a subject.
52. A GLP-1 modulator for use in treating or preventing a disease or condition.
53. Use of an LPAR antagonist in the manufacture of a medicament for use in increasing GLP-1 in a patient.
54. Use of an LPAR antagonist in the manufacture of a medicament for use in treating or preventing a disease or condition characterized by reduced GLP-1 levels or activity.
55. A pharmaceutical composition comprising an LPAR antagonist and a pharmaceutically acceptable excipient, diluent, or carrier, for use in increasing GLP-1 in a patient.
56. A pharmaceutical composition comprising an LPAR antagonist and a pharmaceutically acceptable excipient, diluent, or carrier, for use in treating or preventing a disease or condition characterized by reduced GLP-1 levels or activity in a patient.
57. Use of a modulator of lysophosphatidic acid (LPA) to modulate GLP-1.

As used herein, the terms patient and subject may be used interchangeably.

Methods of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

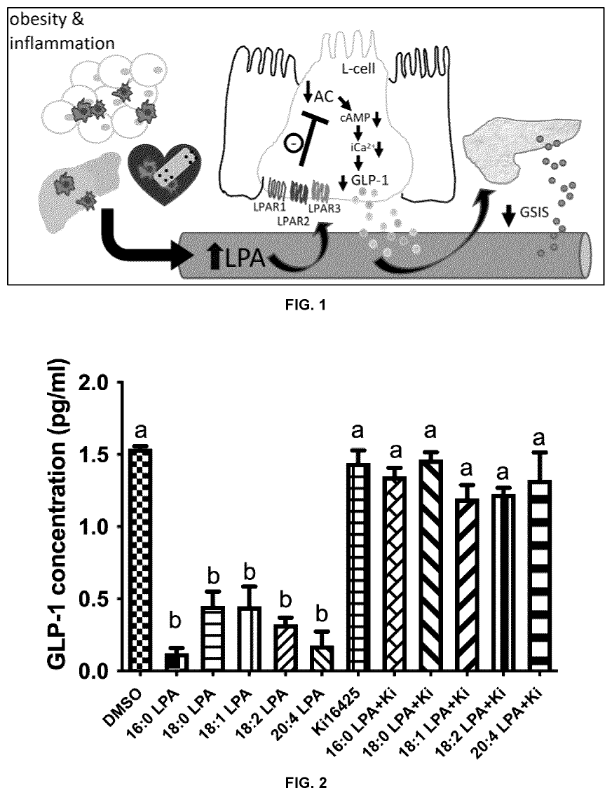

We have found that GLP-1 secretion by L-cells is inhibited by treatment with various LPA species, and that inhibition is prevented by pre-treatment with the LPAR1/2/3 inhibitor Ki16425 (see FIG. 2).

We have grown GLUTag cells in culture to test effects of various LPA species and the LPAR 1/2/3 inhibitor Ki-16425. GLUTag cells are a well characterized L-cell line derived from a mouse intestinal tumour, which have been widely used to study the regulation of GLP-1. Briefly, GLUTag cells were grown until 80-90% confluent. Complete medium, containing fetal bovine serum, was replaced with medium containing charcoal-35 dextran treated serum, which removes lipids and hormones that could interfere with testing. Cells were then treated for 2 hours with medium containing either DMSO as vehicle control (0.1% v/v), 2.5 μM LPA, 10 μM Ki-16425, or a combination of the LPA species and Ki16425, as shown. This work demonstrates that a wide variety of LPA species, including 1-palmitoyl-2-hydroxy-LPA, 1-stearoyl-2-hydroxy-LPA, 1-oleoyl-2-hydroxy-LPA, 1-linoleoyl-2-hydroxy-LPA, and 1-arachidonoyl-2-hydroxy-LPA can all decrease GLP-1 secretion from these cells by over 2/3rds, but that Ki-16425 completely prevents this decrease (FIG. 2). This indicates a direct inhibitory effect of LPA at physiologically relevant levels on GLP-1 secretion by L-cells, and the ability of a commercially available LPAR antagonist to prevent this.

### Example 2

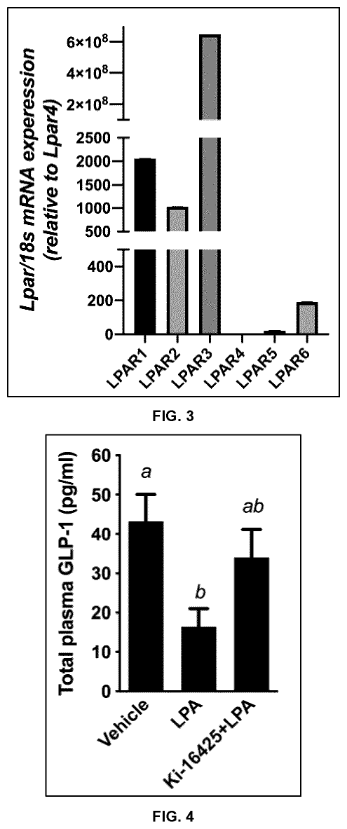

We have found that LPAR 1, 2 and 3 are abundant in GLUTag cells, providing supporting evidence that LPA acted to inhibit GLP-1 through a receptor-mediated effect, and that LPAR antagonists interrupt LPA-mediated inhibitory effects through direct LPAR interaction (see FIG. 3).

We quantified the relative expression of Lpar in GLUTag cells, using cDNA prepared from three separate biological replicates, and have found that LPAR3 is the most highly expressed, with Lpar1 and Lpar2 being second and third. This would suggest that LPAR3 is the receptor that is most highly involved in mediating the inhibitory effect of LPA on GLP-1 secretion. However, LPAR3 is not activated by 1-palmitoyl-2-hydroxy-LPA, or 1-stearoyl-2-hydroxy-LPA, but these lipids clearly inhibited GLP-1 secretion by GLUTag cells, as shown in FIG. 2. This means that it is likely that LPAR1 and LPAR2 are also involved in inhibiting GLP-1 secretion from L-cells. Notably, LPAR1, 2, and 3 all act in association with Gαᵢ inhibitory GCPRs, and therefore all three should be able to act on GLUTag cells to decrease cAMP-regulated GLP-1 secretion, as outlined in FIG. 1. Lpar4, 5, and 6, were present at very low abundance relative to Lpar1, 2, and 3, indicating that the latter group predominate in control of LPA-mediated signalling in L-cells.

### Example 3

We have discovered that injecting mice with one species of LPA (1-oleoyl-2-hydroxy-LPA) decreases circulating GLP-1 levels, and this can be partially prevented by pre-administering the LPAR1/2/3 inhibitor Ki-16425 (see FIG. 4).

We injected mice i.p. with vehicle (10% DMSO in isotonic saline) or 50 mg/kg of 1-oleoyl-2-hydroxy-LPA. Half of the mice injected with LPA were pre-injected with 5 mg/kg Ki-16425 10 minutes prior to receiving LPA. Thirty minutes later, samples of blood were taken by cardiac puncture, and circulating active GLP-1 levels in plasma were analysed by enzyme linked immunosorbent assay. In mice receiving 1-oleoyl-2-hydroxy-LPA, circulating GLP-1 levels rapidly decreased by ˜60%, and this was significantly prevented by pre-administration of the LPAR1/2/3 antagonist Ki-16425.

### Example 4

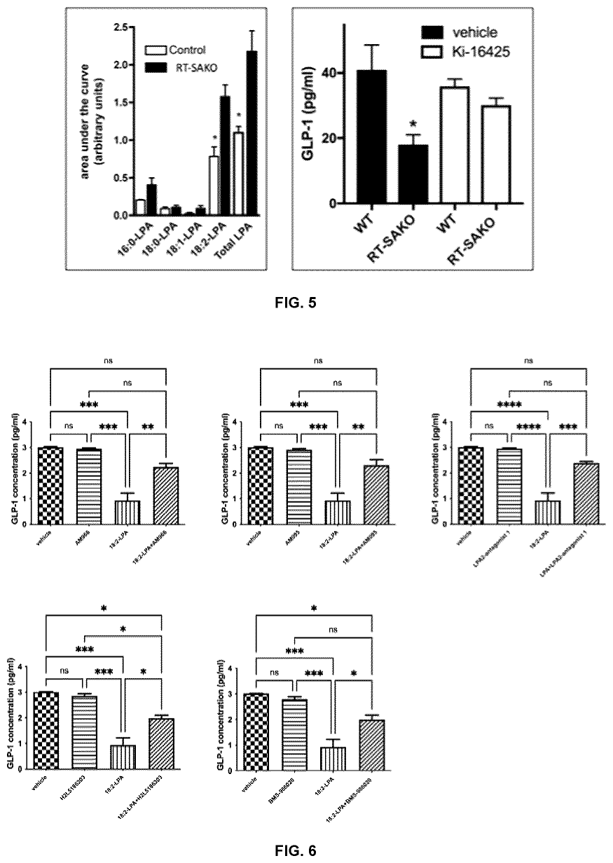

We have determined that GLP-1 levels are lower in a novel genetic mouse model we have created that has elevated circulating LPA. Furthermore, we have found that treatment of these mice with Ki-16425 restores GLP-1 levels (see FIG. 5).

Our laboratory has created a new genetic mouse model to study the isolated effects of obesity on kidney health. This model was not created to test LPA metabolism or to study GLP-1 regulation, but we have made important discoveries on these topics. We have made mice that lack the enzyme adipose triglyceride lipase (ATGL) in renal tubule cells, and have named these mice renal tubule-specific adipose triglyceride lipase knockout (or RT-SAKO) mice. These mice develop fatty kidneys, and as a side effect of changes in kidney cell metabolism, their kidneys make more LPA. As a result, RT-SAKO mice develop elevations in blood lysophosphatidic acid levels by 16-18 weeks of age (FIG. 5, left panel). They also have low circulating levels of GLP-1 (FIG. 5, right panel). We have shown that high LPA causes a reduction in GLP-1, because when we treat RT-SAKO mice with the LPAR1/2/3 blocker Ki-16425, blood GLP-1 levels come back up to close to normal (FIG. 5, right panel). This demonstrates in an in vivo model that elevated circulating LPA, which comes from an endogenous source, can decrease GLP-1 levels, and that an LPAR1/2/3 antagonist can protect against this inhibitory effect of endogenous LPA to rescue GLP-1 levels to normal.

### Example 5

Additional LPAR inhibitors, including AM966, AM095, LPA2-antagonist 1, H2L5186303, and BMS-986020 were tested in GLUTag cells (see FIG. 6).

Experimental Protocol. Briefly, GLUTag cells were grown until 70-80% confluent. Complete medium (low glucose DMEM), containing fetal bovine serum, was replaced with medium containing charcoal-dextran treated serum, which removes lipids and hormones that could interfere with testing. Cells were then treated with medium containing either DMSO as vehicle control (0.1% v/v), 2.5 μM LPA, 10 μM LPAR antagonist, or a combination of the LPA species and inhibitors. For cells treated with both LPA plus LPAR inhibitor: cells were pre-treated with LPA for 30 minutes and then 10 uM LPAR inhibitor was added to the respective wells for a further 2 hours. For cells treated with LPA only, or vehicle only, the total treatment time was 2.5 hours. For cells treated with antagonist only, the treatment time was 2 hours.

Results are shown in FIG. 6. Data were analyzed by 1-way ANOVA with Bonferroni's post-hoc test. Significant differences are as indicated between groups. *P<0.05, **P<0.01, ***P<0.001, ****P<0.00001. ns=not significantly different. Treatment of cells with 18:2-LPA significantly decreased GLP-1 level by >2/3. Treatment of GLUTag L-cells with the LPAR antagonists AM966, AM095, LPA2-antagonist 1, H2L5186303 and BMS-986020 had no significant effect on GLP-1 levels, indicating that these antagonists alone do not alter GLP-1 secretion. The LPAR antagonists AM966, AM095, and LPA2-antagonist 1, caused a complete rescue of GLP-1 secretion, so that GLP-1 levels were not significantly different from control levels in cells treated with 18:2-LPA and any of these inhibitors. The LPAR antagonist H2L5186303 partially rescued GLP-1 levels in wells treated with 18:2. GLP-1 levels were significantly higher in wells treated with H2L5186303 +18:2-LPA than in cells treated with 18:2-LPA alone, indicating a significant beneficial effect of this antagonist on LPA-inhibited GLP-1 secretion. However, GLP-1 levels in wells treated with H2L5186303+18:2-LPA were significantly lower than GLP-1 levels in wells treated with either vehicle alone, or H2L5186303 alone, indicating that the restoration of GLP-1 levels was only partial. The LPAR antagonist BMS-986020 partially rescued GLP-1 levels in wells treated with 18:2-LPA. GLP-1 levels were significantly higher in wells treated with BMS-986020+18:2-LPA than in cells treated with 18:2-LPA alone, indicating a significant beneficial effect of this antagonist on LPA-inhibited GLP-1 secretion. However, GLP-1 levels in wells treated with BMS-986020+18:2-LPA were significantly lower than GLP-1 levels in wells treated with vehicle alone, indicating only a partial rescue to true control levels. However, GLP-1 levels in wells treated with BMS-986020+18:2-LPA were not different than GLP-1 levels in wells treated with BMS-986020 antagonist alone, indicating a fuller rescue to levels of that control group.

Briefly, we tested each of the inhibitors and found that they do not have any effect alone, but they do significantly restore GLP-1 secretion that is reduced by treating cells concomitantly with an LPA species (we chose one that is abundant in blood, and that is elevated in inflammation). Ki16425 appears to have a larger effect than the others (cf. FIG. 2). Without wishing to be bound by any particular theory, it is postulated that this could be because of its pan-receptor effect, or it could just be experimental variation.

We have looked at the LPAR profile in GLUTag cells by qPCR, and LPAR1, 2, and 3 are most highly expressed, and therefore effects of LPA (and by extension, LPAR antagonists) are most likely manifest through these three receptors. All three are G-alpha-i-coupled, and, without being bound by any particular theory, we believe that the most likely mechanism mediating this effect is the inhibition of adenylyl cyclase/cAMP formation, which can stimulate vesicle release.

Ki16425 is an LPAR1/2/3 antagonist, with Ki values of 0.34 uM, 6.5 uM, and 0.93 uM, respectively. Since we treated with 10 uM antagonist, all three receptors would be antagonized by this compound.

AM966 has IC50s (stimulating calcium release in CHO cells) of LPAR1=uM, LPAR2=1.7 uM, LPAR3=1.6 uM, LPAR4=7.7 uM and LPAR5=8.6 uM.

AM095 has IC50s values of ˜0.025 uM for LPAR1, and 10 uM for LPAR2, 3, 4, and 5, so it is more specific for LPAR1.

LPA2-antagonist 1 is selective for LPAR2 with IC50s=17 nM, but IC50s for LPAR1 and 3 being >50 uM.

H2L5186303 is a potent and highly selective LPAR2 antagonist, since it has a Ki=8.9 nM for LPAR2, but Ki concentrations for LPAR1=27 uM, and ˜5 uM for LPAR3.

BMS-986020 IC50s is <0.3 uM for LPAR1.

Another compound of interest is SAR-100842. IC50 concentrations for SARI 00842 range from 59-262 nM for the inhibition of LPA-induced calcium responses in CHO-LPAR1 cell lines.

Combinations of the compounds discussed herein may bring the effective doses lower, or degree of rescue higher.

### Discussion of Examples 1-5

1. Testing of Additional LPAR Antagonists in GLUTag Cells.

FIG. 2 illustrates the inhibitory effect on GLP-1 secretion of various LPA species, and the ability of Ki-16425 to prevent this inhibition. Ki-16425 antagonizes LPAR1, LPAR2, and LPAR3 at Ki values of 0.34 μM, 6.5 μM, and 0.93 μM, respectively. Although it is sometimes considered an LPAR1 antagonist, the close Ki values for LPAR1/2/3 suggest it is a pan-LPAR antagonist for members of this family. Other, more specific antagonists are available. SAR100842 is a compound of interest to inhibit LPAR1 in cultured L-cells. SAR100842 has an IC50 in cells of ˜100 nM for LPAR1, but no activity with LPAR2 or LPAR3 up to 10,000 nM. AM966 is a compound of interest as an LPAR1-specific inhibitor, that has an IC50 of 17 nM in cells with the LPAR1, but an IC50 of 1700 nM with LPAR2 and 1600 nM with LPAR3. Other LPAR1 antagonists of interest include BMS 986020 and AM095. H2L5186303 is of interest, as a potent and highly selective LPAR2 antagonist, since it has a Ki=8.9 nM for LPAR2, but Ki concentrations for LPAR1 and LPAR3 that are ˜3000× and ˜100× higher. ‘LPA2-antagonist 1’ is another selective LPAR2 antagonist, which has an IC50=17 nM with LPAR2, but over 50 μM with LPAR1 or 3, and is of interest. A selective inhibitor of LPAR3 that does not also significantly antagonize LPAR1 is not commercially available, so it is expected that Ki-16425 could be used, and differences between effects of this inhibitor and LPAR1 inhibitors can be taken to infer effects of LPAR3.

2. Testing of Additional LPAR Antagonists, with Additional LPA Species, in Mice.

FIG. 4 shows results demonstrating that pre-injection of mice with the LPAR1/2/3 antagonist Ki16425 significantly abrogates the suppressive effect of 1-oleoyl-2-hydroxy-LPA on circulating GLP-1 levels. It is expected that mice pre-treated with Ki16425, SAR100842, AM966, BMS986200, AM095, H2L5186303, and LPA2-antagonist 1, and then treated with the species of LPA listed in FIG. 2, which are the most abundant LPA species in blood, can be used to determine which drugs have an ability to rescue the inhibitory effects of various LPA species. Combinations of drugs may provide a more complete rescue of GLP-1 secretion. Further experiments in mice are provided in Example 6, wherein the experiment of FIG. 4 (n=3) was repeated with a larger group of mice (see FIG. 8, wherein n=5).

3. Testing of the Ability of LPAR Antagonists to Rescue GLP-1 Secretion in Mice Fed a High-Fat Diet.

In our work on RT-SAKO mice, we have found that LPA levels increase in circulation, while GLP-1 levels decrease, and GLP-1 levels can be partially restored by treating mice with an LPAR1/2/3 antagonist. This demonstrates a role for endogenously produced LPA in the regulation of GLP-1. The finding of increased circulating LPA was unexpected. It is expected that feeding mice a high-fat diet for 8 weeks, to induce obesity, insulin resistance, and an inflammatory state, relative to mice maintained on a control diet could further support the importance of LPAR antagonists as regulators of GLP-1 secretion in vivo. Mice with diet-induced obesity have been found to have elevated circulating LPA, and reduced GLP-1 secretion, although a role for LPA in regulation of GLP-1 has not yet been described outside of the present disclosure. It is expected that treating diet-induced obese mice with each of the LPAR antagonists could restore GLP-1 to control (non-obese) levels, though perhaps to varying degrees of efficacy.

### Example 6

The experimental protocol was as follows:
1. Mice were administered an LPAR antagonist drug, either by oral gavage (for BMS986020, SARI 00842, AM966, AM095, or H2L51863030), or by i.p. injection (for Ki-16425, and LPA2-antagonistl) at the doses indicated in the table below.
2. Control mice were administered only the vehicle (a 10% DMSO solution) given via the same route as the comparison drug was administered.
3. Ten minutes later, mice were injected i.p. either with isotonic saline, or with 50 mg/kg LPA.
4. Thirty minutes later, mice were euthanized by cervical dyslocation, and blood was collected from the heart for preparation of serum that was stored at −80C until it was analyzed for total GLP-1 concentration by ELISA assay using a GLP-1 enzyme immunoassay kit.

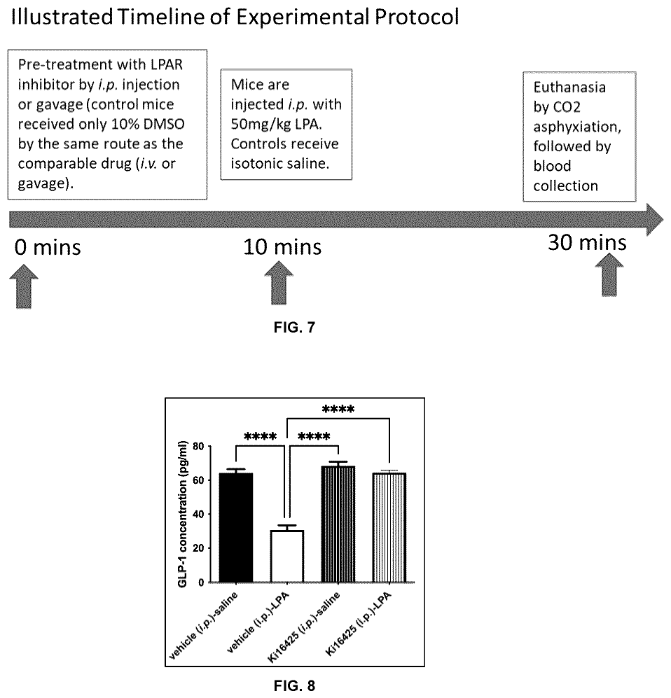

The timeline of the experimental protocol is outlined in FIG. 7, and is summarized in Table 1. For each of the results sections below, 5 mice per group were tested.

TABLE 1
Administration and dose of various LPAR antagonist drugs in mice.

| DRUG | ADMINISTRATION | DOSE |
|---|---|---|
| SAR100842 | GAVAGE | 30 mg/kg |
| BMS986020 | GAVAGE | 30 mg/kg |
| AM966 | GAVAGE | 30 mg/kg |
| AM095 | GAVAGE | 30 mg/kg |
| H2L5186303 | GAVAGE | 30 mg/kg |
| Ki-16425 | IP | 5 mg/kg |
| LPA2-antagonist1 | IP | 5 mg/kg |
| 18:1 LPA | IP | 50 mg/kg |

Results—Ki16425

As shown in FIG. 8, four groups were studied:
(i) Mice injected with 10% DMSO at timepoint 0, then injected with isotonic saline at 10 min.
(ii) Mice injected with 10% DMSO at timepoint 0, then injected with 18:1-LPA (50 mg/kg) 10 min later (dissolved in isotonic saline as vehicle).
(iii) Mice injected with Ki16425 (in 10% DMSO as vehicle) at timepoint 0, then injected with isotonic saline at 10 min.
(iv) Mice injected with Ki16425 (in 10% DMSO as vehicle) at timepoint 0, then injected with 18:1-LPA (50 mg/kg) 10 min later in isotonic saline as vehicle.

GLP-1 concentrations in the serum of mice given a vehicle (10% DMSO) injection i.p. at timepoint 0, followed by isotonic saline i.p. injection, were at a normal, physiological level (FIG. 8).

Injection of vehicle (10% DMSO)-treated mice with LPA caused serum GLP-1 levels to fall by >50% (FIG. 8).

Mice treated with Ki16425 (in 10% DMSO) but not injected with LPA were indistinguishable from mice treated only with the vehicles (i.e. DMSO & saline), indicating that on its own, this LPAR antagonist does not significantly affect blood GLP-1 concentrations (FIG. 8).

However, treatment of mice with Ki16425 completely abrogated the fall in blood GLP-1 levels caused by LPA (see FIG. 8), indicating that Ki16425 protects against decreased GLP-1 levels resulting from excess LPA. In a chronic inflammatory condition where LPA can be elevated, such as diabetes, heart disease, cancer, kidney disease, or others, Ki16425 would therefore be expected to prevent a decline in GLP-1. This should improve blood glucose control, and other conditions where elevated GLP-1 has been found to be beneficial.

Ki16425 acts predominantly on LPAR1 and LPAR3, with respective K, values of 0.34 mM and 0.93 mM, while the K, for LPAR2 is 6.5 mM. Thus, the restoration of GLP-1 blood levels following inhibition by LPA is most likely due to the antagonism by Ki16425 of LPAR1 and LPAR3.

Results— BMS986020

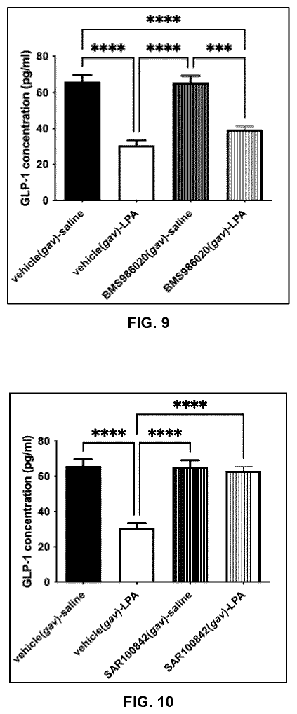

As shown in FIG. 9, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with BMS986020 (in 10% DMSO as vehicle) at timepoint 0, then injected with isotonic saline at 10 min.
(iv) Mice gavaged with BMS986020 (in 10% DMSO as vehicle) at timepoint 0, then injected with 18:1-LPA (50 mg/kg) 10 min later in isotonic saline as vehicle.

GLP-1 concentrations in mice given a vehicle (10% DMSO) gavage at timepoint 0, followed by isotonic saline i.p. injection, were at a normal, physiological level, and very similar to levels seen in mice injected i.p. with 10% DMSO, then given a saline i.p. injection 10 minutes later (FIG. 9).

Injection of vehicle (10% DMSO)-treated mice with LPA caused serum GLP-1 levels to fall by >50% (FIG. 9).

Mice treated with BMS986020 (in 10% DMSO) but not injected with LPA were indistinguishable from mice treated only with the vehicles (i.e. DMSO & saline), indicating that on its own, this LPAR antagonist does not significantly affect blood GLP-1 concentrations (FIG. 9).

Treatment of mice with BMS986020 did not significantly rescue the fall in blood GLP-1 levels caused by LPA, indicating that BMS986020 does not protect against decreased serum GLP-1 levels resulting from excess LPA. This effect was different from the effect observed in cultured GLUTag cells, where BMS986020 did significantly restore GLP-1 secretion.

BMS986020 acts predominantly on LPAR1, with a Ki value of <300 nM. The authors are unaware of any reports of effects on other LPAR (e.g. LPAR2, LPAR3). However, it also has reported activity on bile acid and phospholipid transporters, which might confound its effects on LPAR1 antagonism in vivo, including BSEP (Ki=4.8 mM), MRP4 (Ki=6.2 mM), and MDR3 (Ki=7.5 mM).

Results—SAR100842

As shown in FIG. 10, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with SAR100842 (in 10% DMSO as vehicle) at timepoint 0, then injected with isotonic saline at 10 min.
(iv) Mice gavaged with SAR100842 (in 10% DMSO as vehicle) at timepoint 0, then injected with 18:1-LPA (50 mg/kg) 10 min later in isotonic saline as vehicle.

GLP-1 concentrations in mice given a vehicle (10% DMSO) gavage at timepoint 0, followed by isotonic saline i.p. injection, were at a normal, physiological level (FIG. 10).

Injection of vehicle (10% DMSO)-treated mice with LPA 10 mins later caused serum GLP-1 levels to fall by >50% (FIG. 10).

Mice treated with SAR100842 (in 10% DMSO) but not injected with LPA were indistinguishable from mice treated only with the vehicles (i.e. DMSO & saline), indicating that on its own, this LPAR antagonist does not significantly affect blood GLP-1 concentrations.

However, treatment of mice with SAR100842 completely abrogated the fall in blood GLP-1 levels caused by LPA, indicating that SAR100842 protects against decreased GLP-1 levels resulting from excess LPA. In a chronic inflammatory condition where LPA can be elevated, such as diabetes, heart disease, cancer, kidney disease, or others, SAR100842 would therefore be expected to prevent a decline in GLP-1. This should improve blood glucose control, and other conditions where elevated GLP-1 has been found to be beneficial.

SAR100842 acts predominantly on LPAR1 with a Ki values of 0.1 mM, but has no reported activity on LPAR2 or LPAR3 up to 10 mM. Thus, the restoration of GLP-1 blood levels following inhibition by LPA is most likely due to the antagonism by SAR100842 of LPAR1.

Results—AM966

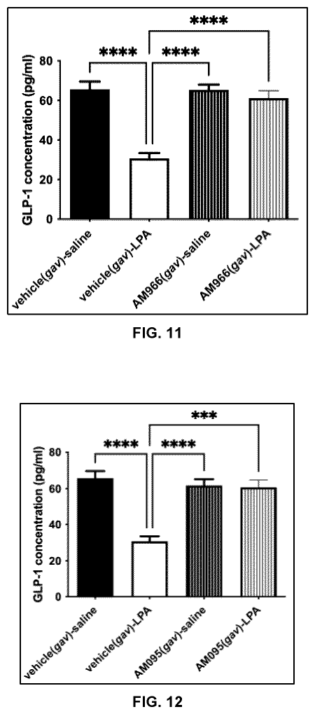

As shown in FIG. 11, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with AM966 (in 10% DMSO as vehicle) at timepoint 0, then injected with isotonic saline at 10 min.
(iv) Mice gavaged with AM966 (in 10% DMSO as vehicle) at timepoint 0, then injected with 18:1-LPA (50 mg/kg) 10 min later in isotonic saline as vehicle.

GLP-1 concentrations in mice given a vehicle (10% DMSO) gavage at timepoint 0, followed by isotonic saline i.p. injection, were at a normal, physiological level (FIG. 11).

Injection of vehicle (10% DMSO)-treated mice with LPA 10 mins later caused serum GLP-1 levels to fall by >50% (FIG. 11).

Mice treated with AM966 (in 10% DMSO) but not injected with LPA were indistinguishable from mice treated only with the vehicles (i.e. DMSO & saline), indicating that on its own, this LPAR antagonist does not significantly affect blood GLP-1 concentrations.

However, treatment of mice with AM966 completely abrogated the fall in blood GLP-1 levels caused by LPA, indicating that AM966 protects against decreased GLP-1 levels resulting from excess LPA. In a chronic inflammatory condition where LPA can be elevated, such as diabetes, heart disease, cancer, kidney disease, or others, AM966 would therefore be expected to prevent a decline in GLP-1. This should improve blood glucose control, and other conditions where elevated GLP-1 has been found to be beneficial.

AM966 acts predominantly on LPAR1 with an IC50 value of 17 nM (calculated based on a calcium assay using CHO cells transfected with the human LPAR1 (Swaney et al, Br. J. Pharmacol. 160(7), 1699-1713 (2010)). The IC50 values for other LPAR are much higher: LPAR2=1700 nM, LPAR3=1600 nM, LPAR4=7700 nM, and LPAR5=8600 nM. Thus, the restoration of GLP-1 blood levels following inhibition by LPA is most likely due to the antagonism by AM966 of LPAR1, although the relatively low IC50 values of LPA with LPAR2 and LPAR3 (in the low micromolar range) also suggests that antagonism of these receptors may be involved.

Results—AM095

As shown in FIG. 12, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with AM095 (in 10% DMSO as vehicle) at timepoint 0, then injected with isotonic saline at 10 min.
(iv) Mice gavaged with AM095 (in 10% DMSO as vehicle) at timepoint 0, then injected with 18:1-LPA (50 mg/kg) 10 min later in isotonic saline as vehicle.

GLP-1 concentrations in mice given a vehicle (10% DMSO) gavage at timepoint 0, followed by isotonic saline i.p. injection, were at a normal, physiological level (FIG. 12).

Injection of vehicle (10% DMSO)-treated mice with LPA 10 mins later caused serum GLP-1 levels to fall by >50% (FIG. 12).

Mice treated with AM095 (in 10% DMSO) but not injected with LPA were not significantly different from mice treated only with the vehicles (i.e. DMSO & saline), indicating that on its own, this LPAR antagonist does not significantly affect blood GLP-1 concentrations.

However, treatment of mice with AM095 completely abrogated the fall in blood GLP-1 levels caused by LPA, indicating that AM095 protects against decreased GLP-1 levels resulting from excess LPA. In a chronic inflammatory condition where LPA can be elevated, such as diabetes, heart disease, cancer, kidney disease, or others, AM095 would therefore be expected to prevent a decline in GLP-1. This should improve blood glucose control, and other conditions where elevated GLP-1 has been found to be beneficial.

AM095 is a potent LPAR1 antagonist because it inhibits GTP-gamma-s binding to Chinese hamster ovary (CHO) cell membranes overexpressing recombinant human or mouse LPAR1 with IC50 values of 0.98 uM and 0.73 uM, respectively.

Results—H2L5186303

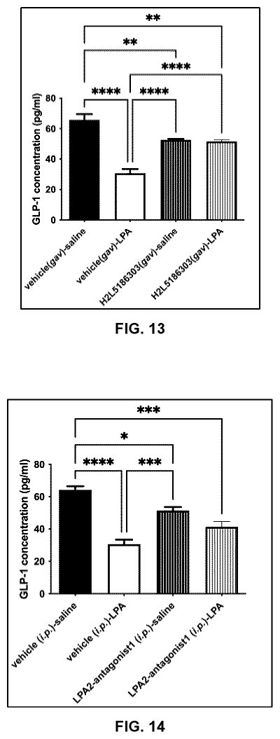

As shown in FIG. 13, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with H2L5186303 (in 10% DMSO as vehicle) at timepoint 0, then injected with isotonic saline at 10 min.
(iv) Mice gavaged with H2L5186303 (in 10% DMSO as vehicle) at timepoint 0, then injected with 18:1-LPA (50 mg/kg) 10 min later in isotonic saline as vehicle.

GLP-1 concentrations in mice given a vehicle (10% DMSO) gavage at timepoint 0, followed by isotonic saline i.p. injection, were at a normal, physiological level (FIG. 13).

Injection of vehicle (10% DMSO)-treated mice with LPA 10 mins later caused serum GLP-1 levels to fall by >50% (FIG. 13).

Blood GLP-1 levels in mice treated with H2L5186303 (in 10% DMSO) but not injected with LPA were slightly, but significantly lower than levels in mice treated only with the vehicles (i.e. DMSO & saline), indicating that on its own, this LPAR antagonist slightly lowered blood GLP-1 levels. This was different than the effect observed in GLUTag cells, where H2L5186303 treatment alone did not significantly affect GLP-1 levels in culture media.

Treatment of mice with H2L5186303 partially rescued the fall in blood GLP-1 levels caused by LPA administration, indicating that H2L5186303 may partially protect against decreased GLP-1 levels resulting from excess LPA. This was similar to effects observed in cell culture, where H2L5186303 partially rescued the reduction in GLP-1 levels that was observed with LPA treatment.

H2L5186303 acts predominantly on LPAR2, with an IC50 value of 8.9 nM, versus 27,354 nM for LPAR1, and 4504 nM for LPAR3 (Fells et al, Bioorg. Med. Chem. 17(21), 7457-7464 (2009)).

Results—LPA2-Antagonist 1

As shown in FIG. 14, four groups were studied:
(i) Mice injected i.p. with 10% DMSO at timepoint 0, then injected i.p. with isotonic saline at 10 min.
(ii) Mice injected i.p. with 10% DMSO at timepoint 0, then injected i.p. with 18:1-LPA (50 mg/kg) 10 min later (dissolved in isotonic saline as vehicle).
(iii) Mice injected i.p. with LPA2-antagonist) (in 10% DMSO as vehicle) at timepoint then injected i.p. with isotonic saline at 10 min.
(iv) Mice injected i.p. with LPA2-antagonist) (in 10% DMSO as vehicle) at timepoint then injected i.p. with 18:1-LPA (50 mg/kg) 10 min later in isotonic saline as vehicle.

GLP-1 concentrations in mice given a vehicle (10% DMSO) by i.p. injection at timepoint 0, followed by isotonic saline i.p. injection, were at a normal, physiological level (FIG. 14).

Injection of vehicle (10% DMSO)-treated mice with LPA 10 mins later caused serum GLP-1 levels to fall by >50% (FIG. 14).

Blood GLP-1 levels in mice treated with LPA2-antagonist) (in 10% DMSO) but not injected with LPA were slightly, but significantly lower than levels in mice treated only with the vehicles (i.e. DMSO & saline), indicating that on its own, this LPAR antagonist slightly lowered blood GLP-1 levels. This was different than the effect observed in GLUTag cells, where LPA2-antagonist) treatment alone did not significantly reduce GLP-1 levels in culture media.

Treatment of mice with LPA2-antagonist) did not significantly rescue the fall in blood GLP-1 levels caused by LPA administration, indicating that LPA2-antagonist) does not offer significant protection against decreased GLP-1 levels resulting from excess LPA. This was also different from effects observed in GLUTag cells in culture, where LPA2-antagonist) significantly increased GLP-1 levels in cells treated with LPA.

LPA2-antagonist) acts predominantly on LPAR2, with an IC50 value of 17 nM, versus >50000 nM for LPAR1 and LPAR3 (Beck et al, Bioorg. Med. Chem. Lett. 18(3), 1037-1041 (2008)).

### Summary of In Vivo Data for Experiment 6

GLP-1 levels were highly similar in mice given the vehicle either by gavage or by i.p. injection, indicating that the route of administration did not affect GLP-1 levels.

GLP-1 levels in mice given vehicle only were similar to those reported in the literature (i.e. physiological).

LPA suppressed blood GLP-1 levels.

GLP-1 was higher in mice treated with LPA+drug versus LPA alone, for every drug except for LPAR2-antagonist) and BMS986020.

Complete rescues of GLP-1 blood levels occurred for Ki16425, SAR100842, AM966, and AM095. All of these predominantly antagonize LPAR1, suggesting that drugs targeting the LPAR1 may be the most effective for blocking the inhibitory effects of LPA on GLP-1 levels. However, antagonism of LPAR3 may also play a role. Our data somewhat supports this, since Ki16425, AM966 and AM095 all antagonize LPAR3 at low micromolar levels.

Although BMS986020 also targets LPAR1, its other activities on bile acid and phospholipid transporters may affect or counteract effects on LPAR1 in vivo. Since this is not yet characterized, conclusions cannot be drawn regarding this drug, and implications for understanding roles for LPAR in GLP-1 secretion.

Although a partial rescue was seen with H2L5186303, it was smaller than for most other antagonists, and may be mediated by partial antagonism of LPAR1 and LPAR3. Taken together with the lack of rescue of GLP-1 levels by the highly-selective LPAR2 antagonist “LPA2-antagonist 1”, this suggests that LPAR2 antagonism is less effective than LPAR1, or LPAR1/3 antagonism at preventing inhibitory effects of LPA on GLP-1 blood levels.

### Example 7: DPP4 Gene Expression and Activity in GLUTag Cells

As discussed above (e.g. see Example 1), experiments have shown that treatment of GLUTag cells with a variety of different species of LPA causes a decrease in GLP-1 levels in the media that those cells are growing in, and that this is rescued by LPAR antagonism. This appears to show that secretion of GLP-1 is decreased by LPA, which makes sense since LPAR are G-protein coupled receptors that can cause changes in secretory pathways, resulting in this rapid effect (i.e. a change in GLP-1 levels that is measurable within 2 hours).

However, this effect (reduced GLP-1 levels in the media around GLUTag cells treated with LPA, that is reversed by LPAR antagonism) could also theoretically occur if DPP4 activity in the cultures is increased by LPA. DPP4 inhibitors are typically peptides that act as competitive substrates for this protease. LPAR antagonists are unlikely to act in this way, as they are lipid mimetics, and LPA is unlikely to act on DPP4 since it is a lipid, not a peptide. Thus, any change in DPP4 activity with LPA treatment, or LPAR antagonist drug treatment, would likely need to be mediated by changes in total DPP4 levels. It is unlikely that, given the timeframe of the effect that we observed (˜2 hours treatment), DPP4 levels could be increased to such an extent to cause the effects observed—(i.e. it is unlikely that LPA could increase DPP4 levels in cells in just 2 hours in a way that would cause a major reduction in GLP-1 levels).

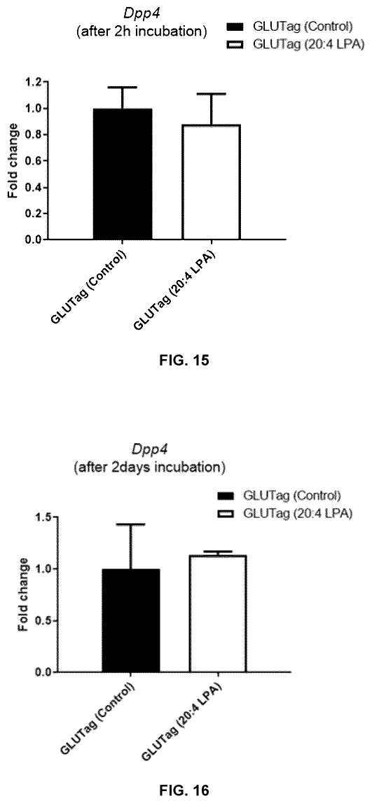

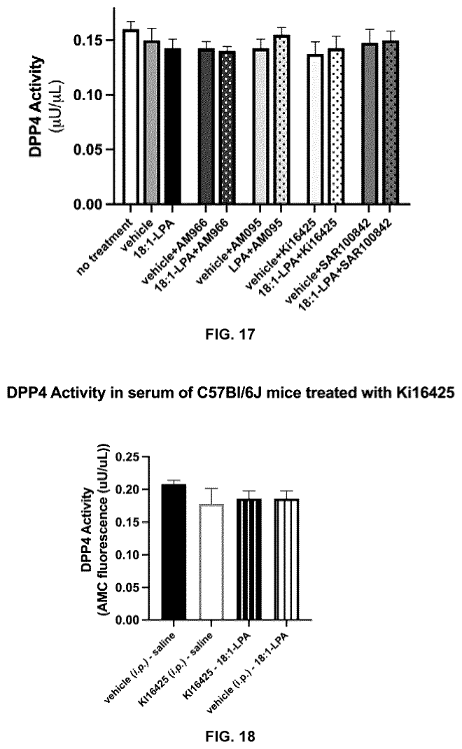

However, since the regulation of DPP4 by LPA and LPAR antagonists is a relevant question, we have investigated this by measuring Dpp4 gene expression in GLUTag cells treated with LPA (FIG. 15 and FIG. 16), and we have also assayed DPP4 activity in cells treated with LPA and/or LPA+LPAR inhibitors (FIG. 17).

DPP4 Gene Expression in LPA-Treated GLUTag Cells

Briefly, GLUTag cells were cultured with 2.5 uM 20:4-LPA, which we have previously disclosed causes an ˜5-fold reduction in GLP-1 levels in the media of cultured GLUTag cells. Cells were harvested after 2 hours, and also after 2 days of incubation, and mRNA was prepared using the Trizol® method, and cDNA was synthesized. Dpp4 gene expression was analyzed by real time PCR using these primers: Dpp4 forward: 5′-GGC CCT GCG TGC TAC TTC CTG GCT CG-3′; Dpp4 reverse: 5′-ACG TCC TGC GCG GCT GCT CTG C-3′, Gapdh forward: 5′-aac ttt ggc att gtg gaa gg-3′; Gapdh reverse: 5′-aca cat tgg ggg tag gaa ca-3′). Gapdh expression was measured as a housekeeping gene, and used to normalize outcomes as a loading control.

Treatment of cells with 20:4-LPA did not significantly change gene expression of Dpp4, even when cells were treated for up to 2 days.

DPP4 Activity in GLUTag Cells Treated with LPA or Combinations of LPA+LPAR Antagonists

Briefly, GLUTag cells were grown to produce the recommended quantity needed for the assay according to the manufacturer's protocol (ab204722 Dipeptidyl peptidase IV Activity Assay Kit (Fluorometric)), and then the wells were treated in media containing charcoal-stripped FBS (10%), with either nothing (no treatment control), or vehicle control (0.05% DMSO), or 2.5 uM 18:1-LPA for 30 minutes, and then the inhibitors (as indicated in the graph of FIG. 17) were added to the vehicle wells and the 2.5 uM 18:1-LPA wells at 10 uM concentrations, as indicated on the graph (FIG. 17), for an additional 2 hours. Cells were then harvested according to the manufacturer's protocol, and lysed using reagents from the kit. Charcoal-stripped FBS was used to remove lipids, including LPA, from serum. Experiments were also carried out wherein the FBS was not stripped with charcoal, and the results were not different.

According to the manufacturer, dipeptidyl peptidase IV (DPP4) Activity Assay Kit (Fluorometric) (ab204722) is an assay where DPP4 cleaves a substrate to release a quenched fluorescent group, AMC (7-Amino-4-Methyl Coumarin), which can be easily detected at Ex/Em=360/460 nm. This assay is rapid, simple, sensitive, and reliable, as well as, suitable for high throughput activity screening of DPP4. This kit detects DPP4 activity as low as 3 ρU per well.

Note that our unnormalized results show all wells with activity >10 pU/well, which is therefore well within the sensitivity range of the assay.

We have reported in data disclosed previously that treatment of GLUTag cells with 18:1-LPA reduces GLP-1 levels by >2/3rds.

In the current assay (see FIG. 17), treatment of cells with 18-1:LPA did not significantly reduce activity of DPP4. Treatment of GLUTag cells with the LPAR antagonists AM966, AM095, Ki16425, and SAR100842 alone also did not significantly affect DPP4 activity, and the combination of 18:1-LPA with these antagonist also did not significantly affect activity.

Thus, changes in GLP-1 levels in response to LPA treatment, or LPAR antagonists, seems most likely to be due to effects on cell GLP-1 levels and secretion, rather than DPP4 activity.

### Example 8: DPP4 Activity in Mice Treated with LPAR Antagonists

The experimental protocol was as follows:
1. Mice were administered an LPAR antagonist drug, either by oral gavage (for SARI 00842 or AM966), or by i.p. injection (for Ki-16425) at the doses indicated in the table below.
2. Control mice were administered only the vehicle (a 10% DMSO solution) given via the same route as the comparison drug was administered.
3. Ten minutes later, mice were injected i.p. either with isotonic saline, or with 50 mg/kg 18:1-LPA.
4. Thirty minutes later, mice were euthanized by cervical dyslocation, and blood was collected from the heart for preparation of serum that was stored at −80C until it was analyzed for total GLP-1 concentration by ELISA assay using a GLP-1 enzyme immunoassay kit.

The timeline of the experimental protocol was the same as the one outlined in FIG. 7, and is summarized in Table 2.

TABLE 2
Administration and dose of various LPAR antagonist drugs in mice.

| DRUG | ADMINISTRATION | DOSE |
|---|---|---|
| SAR100842 | GAVAGE | 30 mg/kg |
| AM966 | GAVAGE | 30 mg/kg |
| Ki-16425 | IP | 5 mg/kg |
| LPA2-antagonist1 | IP | 5 mg/kg |
| 18:1 LPA | IP | 50 mg/kg |

Results—Ki16425

As shown in FIG. 18, four groups were studied:
(i) Mice injected with 10% DMSO at timepoint 0, then injected with isotonic saline at 10 min.
(ii) Mice injected with 10% DMSO at timepoint 0, then injected with 18:1-LPA (50 mg/kg) 10 min later (dissolved in isotonic saline as vehicle).
(iii) Mice injected with Ki16425 (in 10% DMSO as vehicle) at timepoint 0, then injected with isotonic saline at 10 min.
(iv) Mice injected with Ki16425 (in 10% DMSO as vehicle) at timepoint 0, then injected with 18:1-LPA (50 mg/kg) 10 min later in isotonic saline as vehicle.

Injection of vehicle (10% DMSO)-treated mice with 18:1-LPA did not affect DPP4 activity in serum, and the LPAR1/3 antagonist Ki16425 also did not significantly affect DPP4 activity in serum, and combinations of 18:1-LPA with Ki16425 similarly did not affect DPP4 activity in mouse serum. In these same mice, GLP-1 levels were decreased by 18:1-LPA and restored to normal levels by Ki16425. This finding indicates that changes in DPP4 likely are not involved in the changes in GLP-1 levels in serum that have been seen with 18:1-LPA and Ki16425. This further indicates that changes in GLP-1 secretion, rather than breakdown, are most likely responsible for regulation of GLP-1 levels in the blood of these mice.

Results— SAR100842

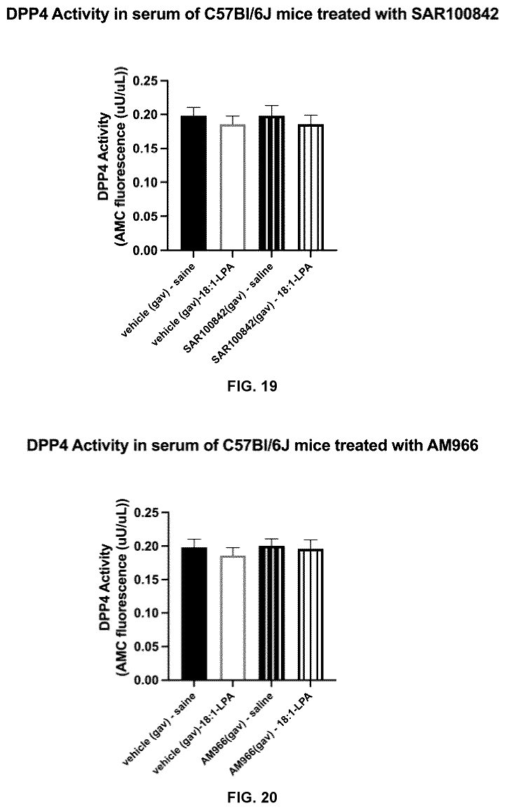

As shown in FIG. 19, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with SAR100842 (in 10% DMSO as vehicle) at timepoint 0, then injected with isotonic saline at 10 min.
(iv) Mice gavaged with SAR100842 (in 10% DMSO as vehicle) at timepoint 0, then injected with 18:1-LPA (50 mg/kg) 10 min later in isotonic saline as vehicle.

Injection of vehicle (10% DMSO)-treated mice with 18:1-LPA did not affect DPP4 activity in serum, and SAR100842 also did not significantly affect DPP4 activity in serum, and combinations of 18:1-LPA with SAR100842 similarly did not affect DPP4 activity in mouse serum. In these same mice, GLP-1 levels were decreased by 18:1-LPA and restored to normal levels by SAR100842. This finding indicates that changes in DPP4 likely are not involved in the changes in GLP-1 levels in serum that have been seen with 18:1-LPA and SAR100842. This further indicates that changes in GLP-1 secretion, rather than breakdown, are most likely responsible for regulation of GLP-1 levels in the blood of these mice.

SAR100842 acts predominantly on LPAR1 with a Ki values of 0.1 mM, but has no reported activity on LPAR2 or LPAR3 up to 10 mM. Thus, the restoration of GLP-1 blood levels following inhibition by LPA is most likely due to the antagonism by SAR100842 of LPAR1.

Results—AM966

As shown in FIG. 20, four groups were studied:
(i) Mice gavaged with 10% DMSO at timepoint 0, then injected with isotonic saline at 10 min.
(ii) Mice gavaged with 10% DMSO at timepoint 0, then injected with 18:1-LPA (50 mg/kg) 10 min later (dissolved in isotonic saline as vehicle).
(iii) Mice gavaged with AM966 (in 10% DMSO as vehicle) at timepoint 0, then injected with isotonic saline at 10 min.
(iv) Mice gavaged with AM966 (in 10% DMSO as vehicle) at timepoint 0, then injected with 18:1-LPA (50 mg/kg) 10 min later in isotonic saline as vehicle.

Injection of vehicle (10% DMSO)-treated mice with 18:1-LPA did not affect DPP4 activity in serum, and AM966 also did not significantly affect DPP4 activity in serum, and combinations of 18:1-LPA with AM966 similarly did not affect DPP4 activity in mouse serum.

In these same mice, GLP-1 levels were decreased by 18:1-LPA and restored to normal levels by AM966. This finding indicates that changes in DPP4 likely are not involved in the changes in GLP-1 levels in serum that have been seen with 18:1-LPA and AM966. This further indicates that changes in GLP-1 secretion, rather than breakdown, are most likely responsible for regulation of GLP-1 levels in the blood of these mice.

AM966 acts predominantly on LPAR1 with an IC50 value of 17 nM (calculated based on a calcium assay using CHO cells transfected with the human LPAR1 (Swaney et al, Br. J. Pharmacol. 160(7), 1699-1713 (2010)). The IC50 values for other LPAR are much higher: LPAR2=1700 nM, LPAR3=1600 nM, LPAR4=7700 nM, and LPAR5=8600 nM. Thus, the restoration of GLP-1 blood levels following inhibition by LPA is most likely due to the antagonism by AM966 of LPAR1.

The lack of effect of LPA or LPAR (Ki16425, SAR100842, and AM966) on DPP4 activity demonstrate that this enzyme, and it's regulation, is not involved in regulation of GLP-1 levels in mouse serum. Thus, changes in GLP-1 secretion, rather than GLP-1 breakdown, are most strongly implicated in changes in GLP-1 that we observed in serum. This agrees quite strongly with data from GLP-1 secreting cells— GLUTags— in culture (cf. Experiment 7).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of modulating glucagon-like peptide 1 (GLP-1) comprising contacting a cell with a modulator of lysophosphatidic acid (LPA).

2. The method of claim 1, wherein the modulator of LPA:
is an inhibitor of LPA signaling or an inhibitor of LPA levels or activity;
is a lysophosphatidic acid receptor (LPAR) antagonist; or
is an LPAR antagonist selected from the group consisting of Ki16425, BMS-986020, SAR 100842, AM966, AM095, H2L5186303, LPA2-antagonist 1 and combinations thereof.

3. (canceled)

4. (canceled)

5. The method of claim 2, wherein the LPAR antagonist is Ki16425.

6. The method of claim 1, wherein the cell expresses LPAR and is capable of secreting GLP-1 under suitable conditions.

7. The method of claim 1, wherein the cell is an L-cell.

8. The method of claim 1, wherein the contacting takes place in vivo.

9. A method of modulating glucagon-like peptide 1 (GLP-1) in a subject comprising administering to the subject a modulator of lysophosphatidic acid (LPA).

10. The method of claim 9, wherein the modulator of LPA:
is an inhibitor of LPA signaling or an inhibitor of LPA levels or activity;
is a lysophosphatidic acid receptor (LPAR) antagonist; or
is an LPAR antagonist selected from the group consisting of Ki16425, BMS-986020, SAR 100842, AM966, AM095, H2L5186303, LPA2-antagonist 1 and combinations thereof.

11. (canceled)

12. (canceled)

13. (canceled)

14. The method of claim 9, wherein the subject has or is suspected of having a disease or condition characterized by reduced or impaired GLP-1 levels or activity, glucose homeostasis and/or insulin-stimulated glucose secretion.

15. The method according to claim 9, wherein modulating glucagon-like peptide 1 (GLP-1) in the subject comprises increasing GLP-1 by:
administering to the subject a therapeutically effective amount of the inhibitor of lysophosphatidic acid (LPA); or
increasing GLP-1 secretion.

16. (canceled)

17. (canceled)

18. (canceled)

19. (canceled)

20. (canceled)

21. The method of claim 14, wherein reduced or impaired GLP-1 levels or activity comprises reduced GLP-1 secretion by L-cells.

22. A method of treating or preventing a disease or condition characterized by reduced GLP-1 levels or activity in a subject, the method comprising administering to the subject a therapeutically effective amount of an inhibitor of lysophosphatidic acid (LPA).

23. The method of claim 22, wherein the inhibitor of LPA is an antagonist of lysophosphatidic acid receptor (LPAR).

24. The method of claim 23, wherein the LPAR is LPAR-1, LPAR-2, LPAR-3, LPAR-4, LPAR-5, and/or LPAR-6.

25. (canceled)

26. The method of claim 23, wherein the LPAR antagonist is selected from the group consisting of Ki16425, BMS-986020, SAR 100842, AM966, AM095, H2L5186303, LPA2-antagonist 1 and combinations thereof.

27. (canceled)

28. The method of claim 22, wherein the disease or condition is diabetes, Type II diabetes, Alzheimer's disease, Parkinson's disease, kidney disease, chronic kidney disease, diabetic nephropathy, a serious renal event, cardiovascular disease, stroke, depression, metal health, pulmonary fibrosis, obesity, aging, or non-alcoholic fatty liver disease.

29. (canceled)

30. (canceled)

31. (canceled)

32. The method of claim 22, wherein reduced GLP-1 levels or activity comprises decreased GLP-1 secretion, decreased GLP-1 secretion from L-cells, decreased GLP-1 production, decreased GLP-1 sensitivity, decreased GLP-1 receptor levels or binding, increased GLP-1 breakdown, excessive GLP-1 inhibition, or a combination thereof.

33. (canceled)

34. The method of claim 22, which further comprises administration of a DPP4 inhibitor, a GLP-1 agonist, or a GLP-1 regulator.

35. (canceled)

36. (canceled)

37. The method of claim 22, wherein the subject is selected for treatment with the inhibitor of lysophosphatidic acid (LPA) by assessing GLP-1 levels or activity in the subject and, if the GLP-1 levels or activity is lower than desired, selecting the subject for the treatment.

38. (canceled)

39. (canceled)

40. (canceled)

41. (canceled)

42. (canceled)

43. (canceled)

44. The method of claim 37, wherein the subject has or is suspected of having a disease or condition selected from the group consisting of diabetes, Type II diabetes, Alzheimer's disease, Parkinson's disease, kidney disease, chronic kidney disease, diabetic nephropathy, a serious renal event, cardiovascular disease, stroke, depression, metal health, pulmonary fibrosis, obesity, aging, or non-alcoholic fatty liver disease.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)
